(19) United States
(12) Patent Application Publication
(10) Pub. No.: US 2005/0107676 A1
(43) Pub. Date: May 19, 2005

(54) Method and apparatus for noninvasive glucose concentration estimation through near-infrared spectroscopy

(76) Inventors: George M. Acosta, Phoenix, AZ (US); James R. Henderson, Phoenix, AZ (US); N. Alan Abul-Haj, Mesa, AZ (US); Timothy L. Ruchti, Gilbert, AZ (US); Stephen L. Monfre, Gilbert, AZ (US); Thomas B. Blank, Chandler, AZ (US); Kevin H. Hazen, Gilbert, AZ (US)

Correspondence Address: GLENN PATENT GROUP, 3475 EDISON WAY, SUITE L, MENLO PARK, CA, 94025, US

(21) Appl. No.: 10/971,447

(22) Filed: Oct. 21, 2004

Related U.S. Application Data
(60) Provisional application No. 60/532,602, filed on Dec. 23, 2003.

(30) Foreign Application Priority Data
Mar. 7, 2003 (WO) PCT/US03/07065

(51) Int. Cl. A61B 5/00

(52) U.S. Cl. 600/316

(57) ABSTRACT

A near-infrared spectrometer-based analyzer attaches continuously or semi-continuously to a human subject and is used to collect spectral measurements of a tissue sample. The spectral readings are used to estimate a biological parameter in the sampled tissue noninvasively, such as glucose concentration. The preferred apparatus is a near-infrared analyzer that includes a base module and a sample module connected together with a communication bundle. The base module contains the bulk of the analyzer, such as a spectrograph and a central processing unit with an algorithm used for converting the optical signal into a glucose concentration. The sample module is typically in a smaller module that interfaces to a tissue sample. The sample module is preferably handheld and provides minimal sampling distortion due to heat or pressure.

### CROSS REFERENCE TO RELATED APPLICATIONS

This application claims benefit of U.S. Provisional Patent Application Ser. No. 60/532,602, filed Dec. 23, 2003; and claims priority from U.S. patent application Ser. No. 10/820,322, filed Apr. 7, 2004, which is a Continuation-in-part of U.S. patent application Ser. No. 09/877,529, filed Jun. 8, 2001, which is a Continuation-in-part of U.S. patent application Ser. No. 09/415,389, filed Oct. 8, 1999, now U.S. Pat. No. 6,411,373 (Jun. 25, 2002); and U.S. patent application Ser. No. 10/472,856, filed Mar. 7, 2003, entitled Compact Apparatus for Noninvasive Measurement of Glucose Through Near-Infrared Spectroscopy, which claims priority from PCT application number PCT/US03/07065, filed on Mar. 7, 2003, which claims benefit of U.S. Provisional Patent Application Ser. No. 60/362,885, filed Mar. 8, 2002; all of which are incorporated herein in their entirety by this reference thereto.

### BACKGROUND OF THE INVENTION

1. Field of the Invention

The invention relates to noninvasive sampling. More particularly, the invention relates to a noninvasive glucose concentration analyzer, which includes a base module, a communication bundle, and a sample module.

2. Description of Related Art

Spectroscopy based noninvasive analyzers deliver external energy in the form of light to a specific sampling site, region, or volume of the human body wherein the photons interact with a tissue sample, thus probing chemical and physical features. A portion of the incident photons are specularly reflected, diffusely reflected, scattered, or transmitted out of the body, they are detected. Based upon knowledge of the incident photons and the detected photons, the chemical and/or structural basis of the sampled site is deduced. A distinct advantage of a noninvasive analyzer is the ability to analyze chemical and structural constituents in the body in a pain-free manner while limiting both consumables and possible generation of biohazards. Additionally, noninvasive analyzers allow multiple analytes or structural features to be determined at one time. Common examples of noninvasive analyzers are those using magnetic resonance imaging (MRI) or x-rays, pulse oximeters, and noninvasive glucose concentration analyzers. With the exception of x-rays, these determinations are performed using relatively harmless wavelengths of radiation. Examples described herein focus on noninvasive glucose concentration determination, but the principles apply to other noninvasive measurements and/or determination of additional blood or tissue analytes.

Diabetes

Diabetes is a chronic disease that results in abnormal production and use of insulin, a hormone that facilitates glucose uptake into cells. While a precise cause of diabetes is unknown, genetic factors, environmental factors, and obesity play roles. Diabetics have increased risk in three broad categories: cardiovascular heart disease, retinopathy, and neuropathy. Diabetics often have one or more of the following complications: heart disease and stroke, high blood pressure, kidney disease, neuropathy (nerve disease and amputations), retinopathy, diabetic ketoacidosis, skin conditions, gum disease, impotence, and fetal complications. Diabetes is a leading cause of death and disability worldwide. Moreover, diabetes is merely one among a group of disorders of glucose metabolism that also includes impaired glucose tolerance and hyperinsulinemia, which is also known as hypoglycemia.

Diabetes Prevalence and Trends

The prevalence of individuals with diabetes is increasing with time. The World Health Organization (WHO) estimates that diabetes currently afflicts 154 million people worldwide. There are 54 million people with diabetes living in developed countries. The WHO estimates that the number of people with diabetes will grow to 300 million by the year 2025. In the United States, 15.7 million people or 5.9 percent of the population are estimated to have diabetes. Within the United States, the prevalence of adults diagnosed with diabetes increased by 6% in 1999 and rose by 33% between 1990 and 1998. This corresponds to approximately eight hundred thousand new cases every year in America. The estimated total cost to the United States economy alone exceeds $90 billion per year. Diabetes Statistics, National Institutes of Health, Publication No. 98-3926, Bethesda, Md. (November 1997).

Long-term clinical studies demonstrate that the onset of diabetes related complications is significantly reduced through proper control of blood glucose concentrations. The Diabetes Control and Complications Trial Research Group, The effect of intensive treatment of diabetes on the development and progression of long-term complications in insulin-dependent diabetes mellitus, N. Eng. J. of Med., 329:977-86 (1993); U.K. Prospective Diabetes Study (UKPDS) Group, Intensive blood-glucose control with sulphonylureas or insulin compared with conventional treatment and risk of complications in patients with type 2 diabetes, Lancet, 352:837-853 (1998); and Y. Ohkubo, H. Kishikawa, E. Araki, T. Miyata, S. Isami, S. Motoyoshi, Y. Kojima, N. Furuyoshi, M. Shichizi, Intensive insulin therapy prevents the progression of diabetic microvascular complications in Japanese patients with non-insulin-dependent diabetes mellitus: a randomized prospective 6-year study, Diabetes Res. Clin. Pract., 28:13-117 (1995).

A vital element of diabetes management is the self-monitoring of blood glucose concentration by diabetics in the home environment. However, current monitoring techniques discourage regular use due to the inconvenient and painful nature of drawing blood or interstitial fluid through the skin prior to analysis, The Diabetes Control and Complication Trial Research Group, supra. As a result, noninvasive measurement of glucose concentration is identified as a beneficial development for the management of diabetes. Implantable glucose analyzers coupled to an insulin delivery system providing an artificial pancreas are also being pursued.

Noninvasive Glucose Concentration Determination

There exist a number of noninvasive approaches for glucose determination. These approaches vary widely, but have at least two common steps. First, an apparatus is used to acquire a reading from the body without obtaining a biological sample. Second, an algorithm converts this reading into a glucose determination.

One species of noninvasive glucose analyzers includes those based upon the collection and analysis of spectra. Typically, a noninvasive apparatus uses some form of spectroscopy to acquire the signal or spectrum from the body. Spectroscopic techniques include but are not limited to Raman and fluorescence, as well as techniques using light from ultraviolet through the infrared [ultraviolet (200 to 400 nm), visible (400 to 700 nm), near-IR (700 to 2500 nm or 14,286 to 4000 cm⁻¹), and infrared (2500 to 14,285 nm or 4000 to 700 cm⁻¹)]. A particular range for noninvasive glucose determination in diffuse reflectance mode is about 1100 to 2500 nm or ranges therein (Hazen, Kevin H. “Glucose Determination in Biological Matrices Using Near-Infrared Spectroscopy”, doctoral dissertation, University of Iowa, 1995). It is important to note, that these techniques are distinct from the traditionally invasive and alternative invasive techniques listed above in that the sample analyzed is a portion of the human body in-situ, not a biological sample acquired from the human body.

Typically, three modes are used to collect noninvasive scans: transmittance, transflectance, and/or diffuse reflectance. For example the light, spectrum, or signal collected is light transmitting through a region of the body, diffusely transmitting, diffusely reflected, or transflected. “Transflected” refers to collection of the signal not at the incident point or area (diffuse reflectance), and not at the opposite side of the sample (transmittance), but rather at some point or region of the body between the transmitted and diffuse reflectance collection area. For example, transflected light enters the fingertip or forearm in one region and exits in another region. When using the near-infrared, the transflected radiation typically radially disperses 0.2 to 5 mm or more away from the incident photons depending on the wavelength used. For example, light that is strongly absorbed by the body such as light near the water absorbance maxima at 1450 or 1950 nm is collected after a small radial divergence in order to be detected and light that is less absorbed, such as light near water absorbance minima at 1300, 1600, or 2250 nm is, optionally, collected at greater radial or transflected distances from the incident photons.

Noninvasive techniques are not limited to sampling the fingertip. Other regions or volumes of the body subjected to noninvasive measurements are: a hand, finger, palmar region, base of thumb, forearm, volar aspect of the forearm, dorsal aspect of the forearm, upper arm, head, earlobe, eye, tongue, chest, torso, abdominal region, thigh, calf, foot, plantar region, and toe. Notably, noninvasive techniques are not necessarily based upon spectroscopy. For example, a bioimpedence meter is a noninvasive device. In this document, any device that reads or determines a glucose concentration from the body without penetrating the skin and collecting a biological sample is referred to as a noninvasive glucose analyzer. Some noninvasive analyzers use invasive methods for purposes of calibration or bias correction of estimated glucose concentration values. Herein, x-rays and magnetic resonance imagers are not considered to be defined in the realm of noninvasive technologies.

There are a number of reports on noninvasive glucose technologies. Some of these relate to general instrumentation configurations required for noninvasive glucose concentration determination while others refer to sampling technologies. Those related to the present invention are briefly reviewed here:

General Instrumentation

Pulse oximeters operate on wavelengths about 660 and 805 nm, which correlate oxy-hemoglobin and deoxy-hemoglobin absorbance bands. Siemens, AG, Verfahren und Great zur kolorimetrischen Untersuchung von Substanzen auf signifikante bestandteile (Method and device for a colorimetric examination of substances for significant components), DE 2,255,300, filed Nov. 11, 1972 describe a pulse oximeter meter operating in a spectral region of 600 to 900 nm, which is shorter than the noninvasive glucose concentration meters of this invention that operate from about 1100 to 2500 nm or ranges therein.

K. Schlager, Non-invasive near infrared measurement of blood analyte concentrations, U.S. Pat. No. 4,882,492, (Nov. 21, 1989) describes a dual beam noninvasive glucose analyzer. This patent is commonly owned with the current application.

R. Barnes, J. Brasch, D. Purdy, W. Lougheed, Non-invasive determination of analyte concentration in body of mammals, U.S. Pat. No. 5,379,764 (Jan. 10, 1995) describe a noninvasive glucose concentration determination analyzer that uses data pretreatment in conjunction with a multivariate analysis to determine blood glucose concentrations.

P. Rolfe, Investigating substances in a patient's bloodstream, UK patent application Ser. No. 2,033,575 (Aug. 24, 1979) describes an apparatus for directing light into the body, detecting attenuated backscattered light, and using the collected signal to determine glucose concentrations in or near the bloodstream.

C. Dahne, D. Gross, Spectrophotometric method and apparatus for the non-invasive, U.S. Pat. No. 4,655,225 (Apr. 7, 1987) describe a method and apparatus for directing light into a patient's body, collecting transmitted or backscattered light, and determining glucose concentrations from selected near-infrared (near-IR) wavelength bands. Wavelengths include 1560 to 1590, 1750 to 1780, 2085 to 2115, and 2255 to 2285 nm, with at least one additional reference signal from 1000 to 2700 nm.

M. Robinson, K. Ward, R. Eaton, D. Haaland, Method and apparatus for determining the similarity of a biological analyte from a model constructed from known biological fluids, U.S. Pat. No. 4,975,581 (Dec. 4, 1990) describe a method and apparatus for measuring a concentration of a biological analyte, such as glucose using infrared spectroscopy in conjunction with a multivariate model. The multivariate model is constructed from a plurality of known biological fluid samples.

J. Hall, T. Cadell, Method and device for measuring concentration levels of blood constituents non-invasively, U.S. Pat. No. 5,361,758 (Nov. 8, 1994) describe a noninvasive device and method for determining analyte concentrations within a living subject using polychromatic light, a wavelength separation device, and an array detector. The apparatus uses a receptor shaped to accept a fingertip with means for blocking extraneous light.

S. Malin, G Khalil, Method and apparatus for multi-spectral analysis of organic blood analytes in noninvasive infrared spectroscopy, U.S. Pat. No. 6,040,578 (Mar. 21, 2000) describe a method and apparatus for determination of an organic blood analyte using multi-spectral analysis in the near-infrared. A plurality of distinct nonoverlapping spectral regions of wavelengths is incident upon a sample surface, diffusely reflected radiation is collected, and the analyte concentration is determined via chemometric techniques. This patent is commonly owned with the current application.

J. Garside, S. Monfre, B. Elliott, T. Ruchti, G. Kees, Fiber optic illumination and detection patterns, shapes, and locations for use in spectroscopic analysis, U.S. Pat. No. 6,411,373, (Jun. 25, 2002) describe the use of fiber optics for use as excitation and/or collection optics with various spatial distributions. This patent is commonly owned with the current application.

Specular Reflectance

R. Messerschmidt, D. Sting Blocker device for eliminating specular reflectance from a diffuse reflectance spectrum, U.S. Pat. No. 4,661,706 (Apr. 28, 1987) describe a reduction of specular reflectance by a mechanical device. A blade-like device “skims” the specular light before it impinges on the detector. A disadvantage of this system is that it does not efficiently collect diffusely reflected light and the alignment is problematic.

R. Messerschmidt, M. Robinson Diffuse reflectance monitoring apparatus, U.S. Pat. No. 5,636,633 (Jun. 10, 1997) describe a specular control device for diffuse reflectance spectroscopy using a group of reflecting and open sections.

R. Messerschmidt, M. Robinson Diffuse reflectance monitoring apparatus, U.S. Pat. No. 5,935,062 (Aug. 10, 1999) and R. Messerschmidt, M. Robinson Diffuse reflectance monitoring apparatus, U.S. Pat. No. 6,230,034 (May 8, 2001) describe a diffuse reflectance control device that discriminates between diffusely reflected light that is reflected from selected depths. This control device additionally acts as a blocker to prevent specularly reflected light from reaching the detector.

Malin, supra, describes the use of specularly-reflected light in regions of high water absorbance such as 1450 and 1900 nm to mark the presence of outlier spectra wherein the specularly reflected light is not sufficiently reduced. This patent is commonly owned with the current application.

K. Hazen, G. Acosta, A. Abul-Haj, R. Abul-Haj, Apparatus and method for reproducibly modifying localized absorption and scattering coefficients at a tissue measurement site during optical sampling, U.S. Pat. No. 6,534,012 (Mar. 18, 2003) describe a mechanical device for applying sufficient and reproducible contact of the apparatus to the sample medium to minimize specular reflectance. Further, the apparatus allows for reproducible applied pressure to the sample site and reproducible temperature at the sample site. This patent is commonly owned with the current application.

Sample Preparation

B. Wenzel, S. Monfre, T. Ruchti, K. Meissner, F. Grochocki, T. Blank, J. Rennert, A method for quantification of stratum corneum hydration using diffuse reflectance spectroscopy, U.S. Pat. No. 6,442,408, (Aug. 27, 2002) describe a method and apparatus for determination of tissue variability, such as water content of the epidermal ridge and penetration depth of incident light. This patent is commonly owned with the current application.

Temperature

K. Hazen, Glucose Determination in Biological Matrices Using Near-Infrared Spectroscopy, doctoral dissertation, University of Iowa (1995) describes the adverse effect of temperature on near-infrared based glucose concentration determinations. Physiological constituents have near-infrared absorbance spectra that are sensitive, in terms of magnitude and location, to localized temperature and the sensitivity impacts noninvasive glucose concentration determination.

Coupling Fluid

A number of sources describe coupling fluids with important sampling parameters.

Index of refraction matching between the sampling apparatus and sampled medium is well known. Glycerol is commonly used to match refractive indices of optics and skin.

R. Messerschmidt, Method for non-invasive blood analyte measurement with improved optical interface, U.S. Pat. No. 5,655,530 (Aug. 12, 1997), and R. Messerschmidt Method for non-invasive blood analyte measurement with improved optical interface, U.S. Pat. No. 5,823,951 describe an index-matching medium for use between a sensor probe and the skin surface. The index-matching medium is a composition containing perfluorocarbons and chlorofluorocarbons.

M. Robinson, R. Messerschmidt, Method for non-invasive blood analyte measurement with improved optical interface, U.S. Pat. No. 6,152,876 (Nov. 28, 2000) and M. Rohrscheib, C. Gardner, M. Robinson, Method and apparatus for non-invasive blood analyte measurement with fluid compartment equilibration, U.S. Pat. No. 6,240,306 (May 29, 2001) describe an index-matching medium to improve the interface between the sensor probe and skin surface during spectroscopic analysis. The index-matching medium is preferably a composition containing chlorofluorocarbons with optional perfluorocarbons.

T. Blank, G. Acosta, M. Mattu, S. Monfre, Fiber optic probe guide placement guide, U.S. Pat. No. 6,415,167 (Jul. 2, 2002) describe a coupling fluid of one or more perfluoro compounds where a quantity of the coupling fluid is placed at an interface of the optical probe and measurement site. Advantageously, perfluoro compounds lack the toxicity associated with chlorofluorocarbons. This patent is commonly owned with the current application.

Positioning

T. Blank, supra, describes the use of a guide in conjunction with a noninvasive glucose concentration analyzer in order to increase precision of the location of the sampled tissue site resulting in increased accuracy and precision in noninvasive glucose concentration estimations. This patent is commonly owned with the current application.

J. Griffith, P. Cooper, T. Barker, Method and apparatus for non-invasive blood glucose sensing, U.S. Pat. No. 6,088,605 (Jul. 11, 2000) describe an analyzer with a patient forearm interface in which the forearm of the patient is moved in an incremental manner along the longitudinal axis of the patient's forearm. Spectra collected at incremental distances are averaged to take into account variations in the biological components of the skin. Between measurements rollers are used to raise the arm, move the arm relative to the apparatus and lower the arm by disengaging a solenoid causing the skin lifting mechanism to lower the arm into a new contact with the sensor head. The Griffith teachings do not suggest the use of a controlled pressure between the forearm sample site and the sampling head. In addition, spectra are not collected during a period of relative motion between the sample and the analyzer.

Pressure

E. Chan, B. Sorg, D. Protsenko, M. O'Neil, M. Motamedi, A. Welch, Effects of compression on soft tissue optical properties, IEEE Journal of Selected Topics in Quantum Electronics, Vol. 2, no. 4, pp.943-950 (1996) describe the effect of pressure on absorption and reduced scattering coefficients from 400 to 1800 nm. Most specimens show an increase in the scattering coefficient with compression.

K. Hazen, G. Acosta, A. Abul-Haj, R. Abul-Haj, Apparatus and method for reproducibly modifying localized absorption and scattering coefficients at a tissue measurement site during optical sampling, U.S. Pat. No. 6,534,012 (Mar. 18, 2003) describe in a first embodiment a noninvasive glucose concentration estimation apparatus for either varying the pressure applied to a sample site or maintaining a constant pressure on a sample site in a controlled and reproducible manner by moving a sample probe along the z-axis perpendicular to the sample site surface. In an additional described embodiment, the arm sample site platform is moved along the z-axis that is perpendicular to the plane defined by the sample surface by raising or lowering the sample holder platform relative to the analyzer probe tip. The U.S. Pat. No. 6,534,012 patent further teaches proper contact between the probe tip and the sample site to be that point at which specularly-reflected light is substantially zero at the water bands at 1950 and 2500 nm. This patent is commonly owned with the current application.

M. Makarewicz, M. Mattu, T. Blank, G. Acosta, E. Handy, W. Hay, T. Stippick, B. Richie, Method and apparatus for minimizing spectral interference due to within and between sample variations during in-situ spectral sampling of tissue, U.S. patent application Ser. No. 09/954,856 (filed Sep. 17, 2001) describe a temperature and pressure controlled sample interface. The means of pressure control is a set of supports for the sample that control the natural position of the sample probe relative to the sample. This patent is commonly owned with the current application.

Data Processing

R. Bames, J. Brasch, Non-invasive determination of glucose concentration in body of patients, U.S. Pat. No. 5,070,874, (Dec. 10, 1991) describe a method of collecting near-infrared noninvasive spectra, preprocessing with an n^{th} derivative, and determining a glucose concentration from the resulting spectrum.

Several approaches exist that employ diverse preprocessing methods to remove spectral variation related to the sample and instrumental variation including normalization, smoothing, derivatives, multiplicative signal correction (Geladi, P., D. McDougall and H. Martens. “Linearization and Scatter-Correction for Near-infrared Reflectance Spectra of Meat,” Applied Spectroscopy, vol. 39, pp. 491-500, 1985), standard normal variate transformation (R. J. Barnes, M. S. Dhanoa, and S. Lister, Applied Spectroscopy, 43, pp. 772-777, 1989), piecewise multiplicative scatter correction (T. Isaksson and B. R. Kowalski, Applied Spectroscopy, 47, pp. 702-709, 1993), extended multiplicative signal correction ⁽H. Martens and E. Stark, J. Pharm Biomed Anal, 9, pp. 625-635, 1991), pathlength correction with chemical modeling and optimized scaling ⁽“GlucoWatch Automatic Glucose Biographer and AutoSensors”, Cygnus Inc., Document #1992-00, Rev. March 2001), and finite impulse response filtering (Sum, S. T., “Spectral Signal Correction for Multivariate Calibration,” Doctoral Dissertation, University of Delaware, Summer 1998; Sum, S. and S. D. Brown, “Standardization of Fiber-Optic Probes for Near-Infrared Multivariate Calibrations,” Applied Spectroscopy, Vol. 52, No. 6, pp.869-877, 1998; and T. B. Blank, S. T. Sum, S. D. Brown and S. L. Monfre, “Transfer of near-infrared multivariate calibrations without standards,” Analytical Chemistry, 68, pp. 2987-2995, 1996). In addition, a diversity of signal, data or pre-processing techniques are commonly reported with the fundamental goal of enhancing accessibility of the net analyte signal (Massart, D. L., B. G. M. Vandeginste, S. N. Deming, Y. Michotte and L. Kaufman, Chemometrics: a textbook, New York: Elsevier Science Publishing Company, Inc., 215-252, 1990; Oppenheim, Alan V. and R. W. Schafer, Digital Signal Processing, Englewood Cliffs, N.J.: Prentice Hall, 1975, pp. 195-271; Otto, M., Chemometrics, Weinheim: Wiley-VCH, 51-78, 1999; Beebe, K. R., R. J. Pell and M. B. Seasholtz, Chemometrics A Practical Guide, New York: John Wiley & Sons, Inc., 26-55, 1998; M. A. Sharaf, D. L. Illman and B. R. Kowalski, Chemometrics, New York: John Wiley & Sons, Inc., 86-112, 1996; and Savitzky, A. and M. J. E. Golay. “Smoothing and Differentiation of Data by Simplified Least Squares Procedures,” Anal. Chem., vol. 36, no. 8, pp. 1627-1639, 1964). The goal of all of these techniques is to attenuate the noise and instrument variation while maximizing the signal of interest.

While methods for preprocessing effectively compensate for variation related to instrument and physical changes in the sample and enhance the net analyte signal in the presence of noise and interference, they are often inadequate for compensating for the sources of tissue-related variation. For example, the highly nonlinear effects related to sampling different tissue locations can't be effectively compensated for through a pathlength correction because the sample is multi-layered and heterogeneous. In addition, fundamental assumptions inherent in these methods, such as the constancy of multiplicative and additive effects across the spectral range and homoscadasticity of noise are violated in the non-invasive tissue application.

Calibration/Analysis

One noninvasive technology, near-infrared spectroscopy, has been heavily researched for its application to both frequent and painless noninvasive measurement of glucose. This approach involves the illumination of a spot on the body with near-infrared electromagnetic radiation, light in the wavelength range of 700 to 2500 nm. The light is partially absorbed and scattered, according to its interaction with the constituents of the tissue. With near-infrared spectroscopy, a mathematical relationship between an in-vivo near-infrared measurement and the actual blood glucose concentration needs to be developed. This is achieved through the collection of in-vivo near-infrared measurements with corresponding blood glucose concentrations that have been obtained directly through the use of measurement tools such as the YSI, HemoCue, or any appropriate and accurate traditional invasive or alternative invasive reference device.

For spectrophotometric-based analyzers, there are several univariate and multivariate methods that are used to develop this mathematical relationship. However, the basic equation which is being solved is known as the Beer-Lambert Law. This law states that the strength of an absorbance/reflectance measurement is proportional to the concentration of the analyte which is being measured as in equation 1,
A=εb C (1)
where A is the absorbance/reflectance measurement at a given wavelength of light, ε is the molar absorptivity associated with the molecule of interest at the same given wavelength, b is the distance (or pathlength) that the light travels, and C is the concentration of the molecule of interest (glucose).

Chemometric calibration techniques extract the glucose related signal from the measured spectrum through various methods of signal processing and calibration including one or more mathematical models. The models are developed through the process of calibration on the basis of an exemplary set of spectral measurements known as the calibration set and an associated set of reference blood glucose concentrations based upon an analysis of fingertip capillary blood, venous, or alternative site samples. Common multivariate approaches requiring a set of exemplary reference glucose concentrations and an associated sample spectrum include partial least squares (PLS) and principal component regression (PCR). Many additional forms of calibration are well known in the art, such as neural networks.

Currently, no device using near-infrared spectroscopy for the noninvasive measurement of glucose has been approved for use by persons with diabetes due to technology limitations that include poor sensitivity, sampling problems, time lag, calibration bias, long-term reproducibility, stability, and instrument noise. Further, current reported versions of noninvasive glucose concentration analyzers do not consistently yield accurate estimations of glucose concentrations in long-term patient trials in the hands of a typical user or professional operator. Fundamentally, however, accurate noninvasive estimation of blood glucose is presently limited by the available near-infrared technology, the trace concentration of glucose relative to other constituents, and the dynamic nature of the skin and living tissue of the patient. Further limitations to commercialization include a poor form factor (large size, heavy weight, and no or poor portability) and usability. For example, existing near-infrared technology is limited to larger devices that do not provide (nearly) continuous or automated measurement of glucose and are difficult for consumers to operate. Clearly, a need exists for a noninvasive approach to the estimation of glucose concentration that provides a long-term accurate and precise glucose concentration estimations in a semi-continuous, continuous or semi-automated fashion.

### SUMMARY OF THE INVENTION

The invention involves the monitoring of a biological parameter through a compact analyzer. The preferred apparatus is a spectrometer based system that is semi-continuously in contact with a human subject and that collects spectral measurements which are used to determine a biological parameter in the sampled tissue. The preferred target analyte is glucose. The preferred analyzer is a near-infrared based glucose analyzer for determining the glucose concentration in the human subject's blood, and body that includes a base module and sample module.

### BRIEF DESCRIPTION OF THE DRAWINGS

FIG. 1 provides a top-down perspective view of a glucose concentration tracking system according to the invention;

FIG. 2 is a block schematic diagram that illustrates relationships between analyzer components according to the invention; and

FIG. 3 provides a perspective view of a glucose concentration tracking system according to the invention.

### DETAILED DESCRIPTION OF THE INVENTION

The following detailed description should be read with reference to the drawings in which similar elements in different drawings are numbered the same. The drawings, which are not necessarily to scale, depict illustrative embodiments that are not intended to limit the scope of the invention.

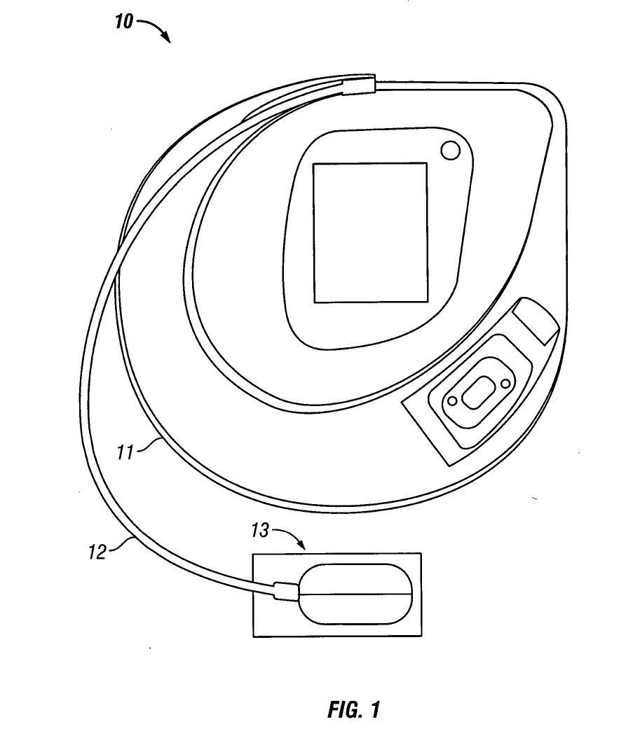

Referring now to FIG. 1, a glucose concentration tracking system (GTS) is presented. The system uses a glucose concentration analyzer that includes at least a source, a sample interface, at least one detector, and a procedure for executing an associated algorithm. Conventionally, all of the components of a noninvasive glucose analyzer are included in a single unit. In FIG. 1, an analyzer 10 is presented in terms of a base module 11, a communication bundle 12, and a sample module 13. The sample module, also referred to as a sampling module, interfaces with a tissue sample and at the same or different times with one or more reference materials. Herein, the combined base module 11, communication bundle 12, sample module 13, and processor is referred to as a spectrometer and/or analyzer 10. Preferably the base module and sample module are in separate housings. Separate housing have benefits including: heat, size, and weight management. For example, the sample module is allowed to be smaller and weigh less without the bulk of the base module. This allows easier handling by the user and less of a physical impact on the sample site by the sample module.

In a first embodiment of the invention, a glucose tracking system (GTS) is used in a clinical setting by a professional. In an alternative embodiment, a glucose tracking system is used in a home setting by an individual. A detailed example of the first embodiment follows.

The glucose tracking system is a medical device that provides noninvasive measurements of blood glucose concentrations in an individual. It is designed for use by healthcare providers in a clinical setting. Blood glucose concentrations are determined through an innovative, noninvasive technique using near-infrared spectroscopy. The glucose concentration tracking system retains measured and calibration blood glucose concentrations in non-volatile memory, such as for the last one month of operation. In a first example, the device accommodates 10,000 blood glucose concentrations that are time, date, and patient stamped. In another example, the glucose concentration tracking system retains processed spectra from the last three days of operation. The device is portable and uses an internal battery supply for normal operation. For longer periods of operation, the device is connected to an alternating current power source and is preferably enabled to charge while operating.

Optionally, for the first and/or last device measurements of the day, a companion glucose concentration reading is obtained using a traditional or alternative site finger-stick glucose concentration meter. The companion concentration is, preferably, transferred automatically to the glucose tracking system through a communication bundle. These companion glucose concentrations are used to calibrate the device and correct for variations in tissue that occur from day-to-day. The individual glucose concentrations are available for display or download to a personal computing device.

Main Assembly

The GTS device is preferably a self-contained unit. The glucose concentration tracking system is, optionally, connected to a personal computing device, such as a personal computer or personal digital assistant, or it may communicate with a server via a network, for example via Bluetooth or 802.11 facilities. For example, the measured blood glucose concentrations are transferred from the device to a workstation running a version of data management software.

Optionally, a private-labeled glucose concentration meter is used to obtain comparison glucose concentrations for the first and last spectra of a session, for any given patient.

The unit includes a main body or base module 11, which houses elements including at least two of: a spectrograph, a battery, associated electronics, coupling fluid, a coupling fluid pump, a reference, at least one detector, a central processor, and a display unit, such as liquid crystal display (LCD) screen or LCD touch screen. A sensor head or sample module 13, which is a portion of the device that contacts a patient's skin, is preferably docked into the main body. The sensor head includes at least two of: a light source, a reflector, one or more optical windows, a thermistor, a temperature control unit, a separate housing from the base module, and tubing for the automatic dispensing of the coupling fluid. The source is preferably a broadband source, infra. The coupling fluid is preferably dispensed in a manner that is transparent to the users. A communication bundle 12 includes at least one of: a fiber-optic strand, an optic, tubing, electrical wiring, and a flexible housing. Optionally, the system contains a stylus, which is used in-lieu of a finger touch on the touch screen.

Optionally, the system uses an arm guide and photo-vasodilating plug to make noninvasive glucose concentration measurements. A guide attachment mechanism is, optionally, incorporated into the final product configuration.

A user interface is used to display information on an output unit, such as a liquid crystal display screen. As a minimum, the screen preferably displays alphanumeric, graphics, and/or simple animation.

The glucose concentration tracking system is preferably battery powered, however for extended operation it is, optionally, connected to an external power converter.

Battery, Battery Charger, and Power Converter

An external power converter converts standard 110/220 volt, 50/60 Hertz alternating current to the appropriate direct current voltage required to charge the internal battery. The charger is preferably internal to the base module. As described, supra, the battery charger is for operation of the glucose concentration tracking system over extended periods. Functionality and performance of the device are preferably not affected by changes in charging states, such as switching from non-charging to charging state. The battery sustains enough charge for use over a testing period, such as about five hours of continuous operations or for ten measurements per day for three days, with each measurement comprising a ten-minute time interval.

Personal Computing Interface

Although the glucose concentration tracking system provides data display on an output device, such as a liquid crystal display touch screen, a personal computing device, such as a personal computer permits a more detailed examination of the data. The personal computer connects to the glucose concentration tracking system through an interface, such as a USB cable, which is used for data transfer. The glucose concentration tracking system device preferably includes software to display patient data values, graph the glucose concentration response profile, and provide relevant statistical analysis. Preferably, the device is capable of outputting a report to a printer.

Separate, enhanced software for the personal computer is, optionally, used to analyze patient data further. Such data are provided in a standard format, such as delimited American Standard Code for Information Interchange (ASCII) text files. The personal computer is used to provide additional functionality. Long-term patient glucose concentration data storage allows detailed analyses for a large population of subjects. Optionally, data transfer is performed using local networks, dial-up, internet communications, or other standard media. Preferably, the glucose concentration tracking system is not configured to collect patient data while a personal computing device is connected.

Functional Groups

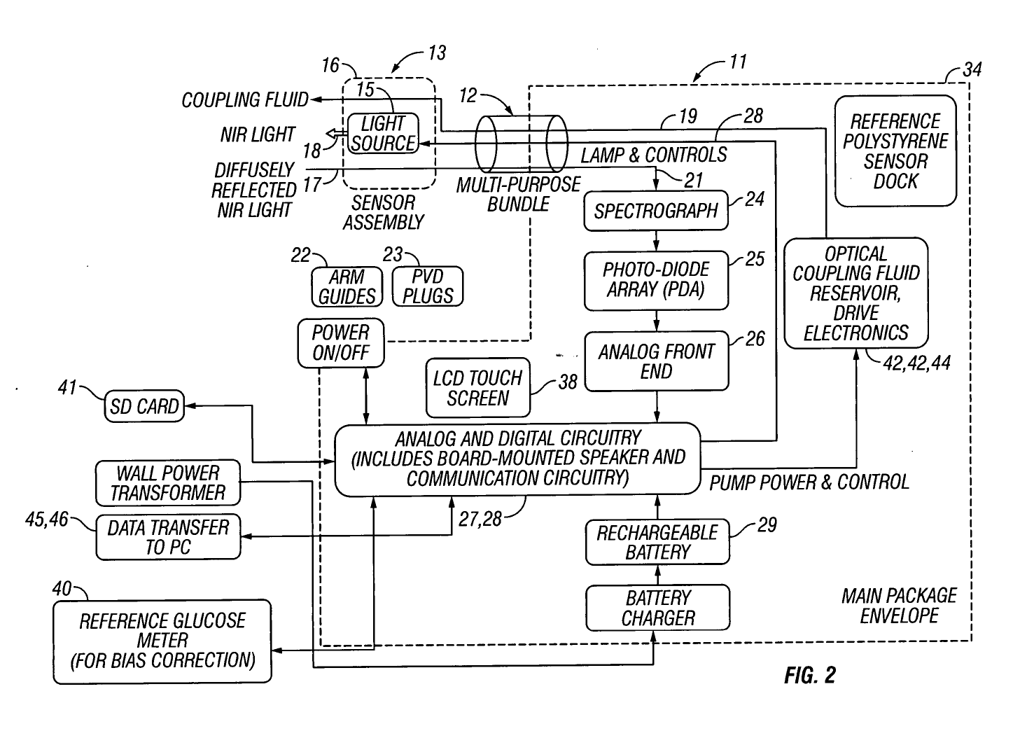

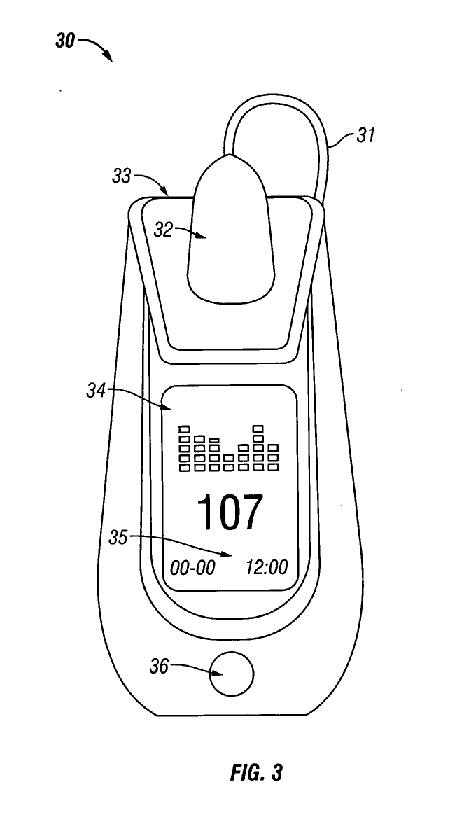

The instrument preferably includes one or more of the following functional groups. FIG. 2 is a block schematic diagram that illustrates relationships between analyzer components according to the invention.
a sensor head 13 that includes a light source 15, the end of the dispensing tube for the coupling fluid 16, a collection fiber 17, and at least one optical window 18;
a bundle assembly 12 containing electrical wires 20, a single fiber-optic strand 21, and a fluid transfer tube 19;
a guide 22 and plug 23, both with and without photo vasodilatation properties;
a spectrograph 24;
a photo-diode array (PDA) 25;
analog front-end (AFE) circuitry 26;
analog circuitry 27,
digital circuitry 28,
a rechargeable battery, 29 performing without a memory effect;
a universal serial bus (USB) communications port 33 (FIG. 3);
a LCD color touch screen display 38;
a LCD touch screen stylus (not shown);
a wavelength reference, such as polystyrene 39;
an intensity reference, such as a polytetrafluoroethylene based reference 40;
an RS-232 communications port 45;
an infrared communications port 46;
a compact flash or SD Card 41;
an optical coupling fluid reservoir 42;
a coupling fluid pump 43;
pump driver electronics 44;
a guide placement mechanism (not shown); and
arm leveling means (not shown).
   GTS Functional Groups

Functional Overview

The following sections describe the top-level functionality and capabilities that are implemented in the glucose concentration tracking system. A preferable relationship of these functional groups is provided in FIG. 2.

Instrument Use

When the GTS is turned on, power is applied to the various internal subsystems. When a component requires a voltage other than that which is supplied by the internal battery, the subassembly that provides the voltage also includes the necessary conditioning circuitry to convert the battery voltage to that which is required.

As necessary, the microprocessor subsystem performs self-tests and system tests to verify that the GTS is ready for use. This always happens on power-up and preferably occurs at least once per day or as necessary. A status message is displayed on the output interface to alert the operator that the self-test is in progress. After successfully completing the self-test, a new message is displayed, which indicates that the GTS is ready for use.

Prior to patient data collection, the operator prepares the patient's sample site, such as a forearm slightly above the wrist, for the analysis. Preparation includes shaving, cleaning, and/or washing of the forearm. Preferably, an arm guide is then adhered to the forearm. An occlusion plug, which, optionally, includes photostimulation means that assists in equalizing forearm glucose concentrations to fingertip glucose concentrations, is then inserted into the arm guide to aid in tissue hydration and/or glucose compartment equalization.

Optionally, upon completion of the preparation of the patient's forearm, the operator selects the patient identification (ID) for the individual to be tested. If no patient ID exists, the operator creates one. Next, the screen prompts the operator to collect a series of measurements to assess guide placement and to establish of a tissue template. A measurement includes optical sampling of tissue, such as a forearm measurement, and using an associated reference, such as a polystyrene spectrum collected within a specified time limit, such as about 2, 5, 60, or 360 minutes. The device evaluates the collected measurements and determines whether the arm guide is placed properly onto the forearm. If proper placement is achieved, the screen prompts the operator to perform a reference glucose concentration measurement within a specified time period or another spectra must be gathered with an associated reference glucose value within an appropriate time frame. After the glucose measurement is complete, the results are transferred to the GTS. Preferably, the transfer is automatic. Typically, a serial interface is used and the transferred data are correlated with a time stamp. The operator then proceeds with the collection of additional measurements at time intervals, such as about fifteen minutes. If the device indicates that the establishment of a tissue template has failed, then the procedure is performed again.

The light source generates the near-infrared light to be used for sampling the sample tissue, such as the dorsal aspect of the forearm. A reflector increases the directed light throughput and reduces some of the unnecessary wavelengths. The lamp output is applied to one or more optical filter elements, which further reduce the content of undesirable light frequencies. The near-infrared light is then projected, through an optical interface on to the tissue site. The interface, optionally, uses a coupling fluid or an optical coupling fluid. Diffusely reflected light from the tissue site is collected by the sensor head and a portion of the light emitting from the sample is directed down at least a single fiber optic strand, which is located in the communication bundle. The communication bundle enters the GTS base module and the fiber optic strand is connected to the spectrograph. The diffusely reflected light is coupled to the spectrograph where it is separated into its spectral components. The appropriate spectral components are then coupled to a detector, such as a photo-diode array (PDA), where a group of photosensitive elements convert the light into corresponding electrical values. The PDA output is an analog signal, which is conditioned and converted into digital format before processing by the microprocessor subsystem in the glucose tracking system.

The digital electronics include a microprocessor, which performs necessary calculations to derive a glucose concentration result from the digital signal. Resulting data are stored in internal memory for later recall.

As described previously, a preferable interface is an LCD screen that includes touch screen capabilities, which are used for option selection and data input. On-screen graphic icons are, optionally, used to assist the user in selecting options or entering data. Fonts are preprogrammed or are selected by the user. Fonts are designed to be read by diabetics that may have vision impairment. Graphical elements, such as icons, are sized such that the image being conveyed is as discernable as textual characters.

Instrumentation

A portion of the analyzer sub-components is described, supra. The following paragraphs add to the above description and provide detail of additional system components.

Sample Module

The sample module includes a sensor head assembly that provides an interface between the glucose concentration tracking system and the patient. The tip of the sample probe of the sample module is brought into contact with the tissue sample. Optionally, the tip of the sample probe is interfaced to a guide, such as an arm-mounted guide, to conduct data collection and it is removed when the process is complete. Guide accessories include an occlusion plug that is used to fill the guide cavity when the sensor head is not inserted in the guide, and/or to provide photostimulation for circulation enhancement. In one example, the following components are included in the sample module sensor head assembly: a light source, a single fiber optic, and coupling fluid. These elements are described, infra.

Sensor Head Assembly

The sensor head assembly is interfaced with the tissue sample. Light from the sensor head illuminates the tissue sample site, such as at the center of a guide on the forearm. The sensor head assembly couples near-infrared energy coming from the light source and disperses it over the target tissue area. A portion of the diffusely reflected light from the tissue is collected by the sensor head into at least one fiber-optic strand for transmission to the spectrometer. Preferably, a coupling fluid is used to maximize energy transfer between the GTS sensor head and the sampled tissue.

Light Source

The near-infrared light source used on the GTS includes at least one of: a lamp, a reflector, an optic, and one or more optical filters. The lamp is preferably a gas-filled tungsten-filament lamp that provides the optical energy to be projected on the tissue site. The reflector increases the directed polychromatic light output from the source to the tissue sample. One or more optical filters are designed to restrict the wavelengths of light that reach the tissue. The filters are preferably anti-reflection coated, have high and constant transmission characteristics in the spectral region of interest, and reduce light outside of the spectral region of interest, which is about the range or 1200 nm to 1850 nm. Preferably, the transmission properties of the filter have minimal change during the measurement period and do not exhibit significant passband ripples, such as exceeding five percent. Examples of filters include longpass, shortpass, and/or bandpass filters.

Guide

A guide is an optional component designed to aid in repeatably positioning the tip of the sample probe of the sample module in relation to the sample tissue volume. The design of the guide incorporates ergonomic and human factors considerations, such as accommodating a size range from the 5% 13-year old female to the 95% adult male. The guide is attached to the individual at a selected tissue site. A guide kit preferably includes a disposable guide with an adhesive patch attached to the underside thereof and peel-off backing, arm attachment instructions, and a plug for occluding and/or photostimulating the tissue sample site when the sensor head is removed.

The arm guide is to be placed onto the tissue sample site in an easy, reproducible, and proper manner, such that minimal interpretation or guesswork is required from the operator or user. Preferably, the guide creates a positive meniscus of skin tissue at the sample site. The guide is optionally used to distribute the weight of the sample probe about the sample site through the use of larger area and/or with flexible material.

Coupling Fluid

Coupling fluid is optionally used between the tip of the sample probe and the sampled tissue volume. In one example, the coupling fluid is delivered via a tube from a reservoir integrated into the analyzer. In another example, the temperature of the coupling fluid approximates the skin temperature of the sample site when it leaves the sensor head assembly and flows onto the skin. Examples of coupling fluid include: optical coupling fluid, refractive index matching coupling fluid, coupling fluid, a fluoropolymer, FC-40™ (3M Company, St. Paul, Minn), FC-70™ (3M Company, St. Paul, Minn.), a perfluorocarbon, a fluorocompound, a chlorofluorocarbon, and the like. Any of these is optionally mixed with an additive or each other to form additional coupling fluids.

Reference Materials

Reference materials, such as polystyrene and/or a Labsphere™ (Labsphere, Inc, North Sutton, N.H.) reference, used during measurement operations are preferably integrated into the GTS. In one example, the device prompts the operator whenever a wavelength reference is necessary. In another example, an intensity reference spectrum is periodically gathered while the sensor is docked in the main instrument housing.

Communication Bundle

The communication bundle is a multi-purpose bundle. The multi-purpose bundle is a flexible sheath that includes at least one of:
electrical wires to supply operating power to the lamp in the light source;
thermistor wires;
one or more fiber-optics, which direct-diffusely reflected near-infrared light to the spectrograph;
a tube, used to transport optical coupling fluid from the base unit, through the sensor head, and onto the measurement site;
a tension member to remove loads on the wiring and fiber-optic strand from pulls; and
photo sensor wires.
   Preferably, the bundle has labeling instructions to the user to train the user not to twist the bundle and, optionally, mechanical means to prevent it from twisting more than one-quarter turn in either direction.
   Base Module

A portion of the diffusely reflected light from the site is collected and transferred via at least one fiber-optic, free space optics, or an optical pathway to the spectrograph. The spectrograph separates the spectral components of the diffusely reflected light, which are then directed to the photo-diode array (PDA). The PDA converts the sampled light into a corresponding analog electrical signal, which is then conditioned by the analog front-end (AFE) circuitry. The analog electrical signals are converted into their digital equivalents by the analog circuitry. The digital data are then sent to the digital circuitry where they are checked for validity, processed, and stored in non-volatile memory. Optionally, the processed results are recalled when the session is complete. After additional processing, the individual glucose concentrations are available for display or transfer to a personal computer. Details of some of the base module components are provided, infra.

Spectrograph

The GTS contains a spectrograph, which is used to separate collected light into its spectral components. In one example, a single fiber-optic strand is positioned at the entrance slit of the spectrograph. The output from the spectrograph is then coupled on the light sensitive elements of the photo-diode array (PDA).

Photo Diode Array

The PDA is preferably an extended InGaAs array or equivalent, preferably with a sensitivity range of greater than fifty percent from about 1200 nm to 1850 nm. In one example, the PDA has a minimum detector density of 250 elements. Typically, the PDA has a minimal number of allowed dead pixels. The low-level signal-processing module is, preferably, capable of assessing and compensating for additional dead pixels as they occur.

Analog Front End (AFE) Electronics

The AFE electronics include at least one of:
signal conditioning of the PDA temperature signal;
signal conditioning for the analog outputs from the PDA; and
signal transfer to the main electronics

Analog Electronics

The analog circuitry controls the data acquisition and signal processing for the analog output from the PDA, provides the lamp driver output used to activate and deactivate the lamp, and/or provides temperature readings. Spectral data are transferred to the digital circuitry from the analog circuitry.

Digital Electronics

The microprocessor subsystem, which is located on the digital circuitry, receives data from the analog circuitry and calculates an interim absorbance spectrum. It then stores interim data and, when the session is complete, performs the bias corrections on individual readings, calculates the glucose concentration, and makes the results available for display or transfer to a personal computing device.

The microprocessor subsystem also provides overall system control and timing functions. In addition, it provides the informational interface to the operator, such as through the LCD touch screen. Self-test functions are performed by the microprocessor to ensure that its subsystem and other subsystems of the GTS are functioning properly.

Microprocessor Hardware

The hardware associated with the microprocessor subsystem is, preferably, capable of performing the required processing in the allotted time, without interfering with other operations. For example, the microprocessor subsystem runs a Linux operating system. Preferably, the microprocessor itself is a high-performance, 32-bit unit, which has a proven track record in other portable, digital devices, such as cellular phones and communications equipment. Any support functionality, such as infrared data transfer and universal serial bus support is also controlled by the microprocessor. In this example, the design uses non-volatile random access memory in place of a hard disk storage system.

The hardware performs the power-up self-tests necessary to ensure that the microprocessor subsystem is fully functional. If any of these tests fail, the hardware generates an error condition, which prevents normal device usage.

Software

The software of the GTS provides multiple functions preferably including:
device initiation tests and standardization files creation;
self-tests (Build and Install) and quality control tests, to verify that the applicable subsystems within the GTS are functioning properly, that are performed as frequently as necessary;
system operations such as; input/output control, display generation, timing/sequencing, and status monitoring;
dedicated processing such as:
   system performance checks;
   signal filtering;
   signal conditioning;
   error checking;
   blood glucose concentration calculation;
   identification of incorrect spectra; and
   calculation of an absorbance spectrum;
temperature sensors;
guide placement assessment (screening module);
tissue template calculation;
high level processing module;
invalid tissue state (physiological or mechanical changes);
low coupling fluid levels;
data transfers to a personal computer; and
electronic and manual data capture from a reference meter, such as a FreeStyle™ (TheraSense, Alameda, Calif.) or a HemoCue™ (HemoCue, Ltd. UK).

External Power Converter

The external power converter for the GTS is preferably a standard alternating current adapter, which converts wall power to system power level requirements. The GTS, optionally, operates while the battery charger is operating with no degradation in performance of the device as it relates to noninvasive blood glucose concentration monitoring.

Touch Screen

A touch screen is preferably used for control selection and data input by the user. The touch screen responds to pressure applied to the liquid crystal display (LCD) and provides the embedded computer with the on-screen location for the point of pressure. Low-level driver software provides an interface between the touch screen data and the embedded computer.

The LCD screen preferably has a minimum display resolution of 320×240 pixels and supports 256 colors. The display provides sufficient illumination, such that it is easily read in a brightly lit clinical environment.

System and Performance

The following sections provide preferable operational specifications for the glucose concentration tracking system.

User Characteristics

The GTS analyzer requires some level of operator and/or patient qualification. Examples of training include: successfully complete a training program, watching a training video, reading a manual, demonstration of sample probe placement, and/or following instrument screen directions. In a professional setting, the operator uses the GTS on a subject or patient to determine one or more glucose concentrations. In another example of a setting, such as a home, the subject or patient operates the GTS analyzer in the absence of an operator.

Subject Characteristics

The patient eligibility characteristics are determined by a government regulatory body, a prescribing doctor, or by other healthcare professionals. Further considerations include:
age, such as 13 years or older; and
a suitable sample site, such as a forearm, on which the guide is located, and a site free of tissue anomalies that interfere with analysis, such as burns, abrasions, lacerations, growths, tattoos, and skin conditions.

Minimal Setup Procedures

The GTS is designed to require minimal setup procedures.

System Warm-up Time

The system warm-up time is preferably minimized and is typically measured from the point that the operator turns on the GTS until it indicates that it is ready to operate. The warm-up time is minimal, such as less than about one minute. One method of monitoring stabilization is though periodic scans of a reference material measured against a specification.

Data Collection Time

The actual time required to accomplish the data collection needed to generate a blood glucose concentration is minimal, such as less than about fifteen seconds. The resulting glucose concentration is either displayed or is stored in memory. The overall procedure time is typically limited to three minutes with one minute being typical from the time the patient sits down until he stands up again. As necessary, an error message is displayed if any of these time limits are exceeded.

This time limit is not intended for initial setup, such as guide placement, a testing procedure, or a calibration procedure.

Calibration Procedure

In the professional device, following the first and last patient spectra in a test series for a single patient a reference blood glucose concentration is preferably used. For personal use, one, both, or neither of these reference glucose concentrations is required. Software preferably captures the time stamp of the reference blood glucose concentration determination. This concentration is preferably automatically transferred from a reference glucose concentration meter. The calibration blood glucose concentration must be in the range of the GTS analyzer, such as between 50 mg/dL and 350 mg/dL. Optionally, the device is capable of accepting reference blood glucose concentrations from other reference meters, such as a HemoCue™, during clinical trials.

Parameter Range

The analyzer measures blood glucose concentrations over a range, such as 50 to 350 mg/dL. For measured values that fall within this range, the GTS processes and stores the concentrations as described, supra. Calculated glucose concentrations that fall outside of this range are recorded, but are displayed with a message, such as LLL for glucose concentrations below 50 mg/dL, and HHH for glucose concentrations above 350 mg/dL.

Visual and Audible Alarm Indicators

The GTS preferably alerts the operator of error conditions with on-screen messages and/or audible alarms. Error conditions include at least one of:
a power indicator;
instrument malfunction, including out of range instrument performance characteristics;
a low battery charge;
inappropriate operating environment, such as a temperature outside of defined limits;
improper sensor head or guide placement;
a calibration reference value transfer failure;
exceeding a time limit;
identification of an invalid or incorrect material;
a reference glucose concentration and collection of a tissue spectrum not performed within a time interval relative to each other;
a lamp status error, such as burned-out or abnormally low or high output;
a sampling error, such as skin temperature out of range;
a quality control/self-test error;
a plug dislodgement; and
a photostimulation failure, such as a dead battery.

Usability

In a professional setting, the GTS is intended to be operated by a trained healthcare professional. The following characteristics are designed to enhance the device's usability:
the GTS is preferably usable on both right and left-handed individuals;
the operator is able to accomplish all data collection-related activities within the allotted time;
error conditions are readily identifiable so that the operator can repeat the measurement or resolve the problem;
gathering reference spectra is preferably automated and transparent to the operator; and
selecting and running an analysis preferably requires less than three touch screen prompts and/or selections.
   Sensor Head-to-Patient Related Items

Guide Placement

A guide is an optional component of the analyzer as described, supra.

In one embodiment, the GTS is used in a professional environment to conduct specific blood glucose concentration testing. As such, a guide is only attached to the patient while at the healthcare facility. The guide is intended to remain attached throughout the visit to the facility, with minimal guide movement relative to the tissue site.

If the guide does require repositioning/re-attachment, it is typically necessary to restart the test procedure, including the calibration glucose concentration determination. The guide is secured by a self-adhesive patch.

A self-placing device is preferably used to minimize the risk that the guide would need repositioning. For a guide with an aperture, the tissue within the aperture is planar with the guide, has a small positive meniscus into the guide aperture, or has a small negative meniscus within the guide aperture.

Arm-to-Sensor Head Pressure

Due to the guide design, there is minimal pressure exerted between the tip of the sample probe and the tissue sample. The pressure at the measurement site is preferably limited by the protrusion of the optical interface of the sensor head into the tissue measurement site.

Sample Temperature

The patient skin temperature at the sample site is preferably in the range of about 85 to 100 degrees Fahrenheit and more preferably in the range of about 90 to 95 degrees Fahrenheit at the time of measurement. The temperature at the tip of the sensor head is preferably controlled to be within the range of about 90° to 95° F. The software module is used to detect when the skin temperature is outside this range and is detrimental to the measurement. The external temperature of the sensor head is typically held below 104° F.

The temperature of the optical coupling fluid as it leaves the sensor head is preferably within the range of 90° to 95° F. Optionally, the optical fluid is heated passively, which reduces power requirements and allows, for example, a smaller capacity battery.

Tissue Recognition

In another embodiment of the invention, after the data collection process is initiated, the display prompts the operator to place the sensor head onto the patient's forearm. The GTS then detects when the sensor head is in contact with the sample site and initiates data collection based upon the spectra. In addition, the GTS preferably detects and identifies any changes in the tissue template.

Ergonomic Range

The ergonomic range for the guide preferably accommodates a wide number of body types. For example, female size characteristics consistent with the range defined by the region above five percent for the female population and below ninety-five percent for the male population. In this example, male sizes which are less that the 5% female cutoff point are considered out of range, as are female characteristics that are above the 95% male cutoff point.

Coupling Fluid

A coupling fluid is preferably used with the GTS analyzer. A specific type of coupling fluid known as optical coupling fluid is alternatively used with the GTS analyzer. The GTS specifications preferably include an coupling fluid that:
does not readily evaporate;
is near-infrared inactive;
is readily dispensed;
is non-toxic and does not irritate skin tissue;
does not emit harmful fumes within storage and operating temperatures, where operating temperatures include both environmental and component temperatures;
is nonflammable;
is replaceable/refillable by the operator without special tools or considerations; and
is contained in a fluid reservoir that does not require replacement more than one time per day.
   In addition, mechanical fluid management is designed to prevent damage of the GTS in the event of an accidental spill. In addition, the instrument preferably warns the operator of a low or minimal remaining coupling fluid condition.

Photostimulation

An optional photostimulation device is used to help to equilibrate glucose concentration in the sampled tissue with capillary or venous blood glucose concentrations. The photostimulation is, preferably, integrated into the guide plug and optically covers the sampled tissue surface. The preferred photostimulation plug is at least one of:
irradiated the sample site during periods of non-measurements;
biocompatible;
in contact with the sample site and simulates sensor head pressures;
provides and couples approximately 850 to 950 nm light and preferably 890 nm or 910 nm light to the measurement site;
used to stimulate circulation through nitric oxide production and results in equalization between the forearm glucose concentration and fingertip glucose concentration;
powered by rechargeable batteries that last for at least one day before recharging; and
designed for use with an off-the-shelf battery recharger.

Intensity Reference

A reference is used to collect background lamp intensity. Preferably, the reference is at least one of:
integrated into the system;
immutable;
non-near-infrared absorbing;
hydrophobic;
homogeneous;
spatially robust with regards to position;
undetectable stray light;
repeatable; and
diffusely reflective.

Wavelength Reference

A wavelength reference is used to establish and/or verify the wavelength axis of the GTS analyzer. One suitable wavelength reference is polystyrene. Preferably, the wavelength reference is at least one of:
used to perform wavelength standardization;
integrated into the system;
immutable;
hydrophobic;
homogeneous;
spatially robust with regards to position; and
repeatable.

Stability

Stability refers to the ability of the GTS instrument to remain in a functional and calibrated state over a given period, thereby allowing the production of consistently accurate and precise glucose concentration estimations. Because GTS glucose readings are correlated and/or adjusted to reference glucose concentrations on a periodic basis, the GTS demonstrates adequate stability between reference readings.

Portability

Portability refers to the instrument's stability remaining constant after moving the instrument from one location to another. The current GTS is designed to be portable from one given location to another without the movement affecting the stability of the unit.

Maintainability

The GTS is designed so that most maintenance items, including preventative maintenance procedures, are accomplishable by the operator or user. For example, the analyzer is designed for simple sensor head replacement, lamp replacement, and/or battery replacement.

Data Transfer

The GTS is preferably capable of internet connectivity to allow for measurement related data transfer, remote diagnostics, and software upgrade.

Multiple Patient/Operator Requirements

The GTS is, optionally, used non-simultaneously with multiple subjects and therefore preferably capable of using and/or creating patient information, such as operator IDs, unique subject identifications, patient data files, and folders. As a security precaution, the software preferably locks out deletion of any patient data from the last 30 days of measurement or system configuration files.

Device Cleaning

The sample probe tip is preferably designed to allow cleaning between subjects and between measurements. Cleaning includes any of wiping of the sensor head with a wet cloth, absorbent tissue, an oil-free tissue, damp cloth, an alcohol swab, and/or a mild cleaning solution, such as a ten percent bleach solution.

Mean Time between Failure

The mean time between failure for components, other than the lamp and batteries, preferably exceeds five years. It is understood that the coupling fluid reservoir requires replacement or refilling to replenish the fluid that is used in normal operation.

Analytical Performance

The GTS is designed to provide performance characteristics that will allow the device to be approved by the United States Food and Drug Administration and the European Union. Example performance characteristics include:
a measurement range of 50 to 350 mg/dL;
a measurement bias versus a reference of:
   ≦20 mg/dL from 50 to 100 mg/dL; and
   ≦20% above 100 mg/dL;
a median standard deviation of:
   15% over a range of 50 to 100 mg/dL;
   ≦12% over a range of 101 to 150 mg/dL; and
   ≦10% over a range of 151 to 350 mg/dL.
      Physical Specifications

The following sections provide example mechanical and electrical specifications for the glucose concentration tracking system.

Physical Characteristics

The GTS is preferably a portable, professional-use device.

Dimensions

The GTS is designed to be as small as possible. The GTS, preferably, has external dimensions not to exceed 7 inch×4 inch×2 inch representing the smallest device size attainable with currently available components. These dimensions are subject to revision as smaller commercial or custom parts become available.

Device Weight

The weight for the GTS is preferably less than about two pounds.

Sensor Head Weight

The weight of the sensor head is typically under 60 grams and is preferably as light as possible, such as in the range of 27 to 30 grams. Alternative embodiments have sensor heads weighing about 40 or 50 grams.

Shipping Weight

The estimated weight of the instrument with accessories and operator's manual is preferably less than ten pounds.

Operator-Instrument Interface

There are two types of operator-instrument interfaces: 1) inputs to the GTS by the operator and 2) outputs from the GTS to the operator. Examples of inputs to the GTS include touch screen selections, such as data input through the touch screen, or control inputs, such as the on/off switch. Examples of outputs from the GTS include on-screen menus, options, messages, and audio prompts, such as beeps.

Patient Instrument Interface

The patient-instrument interface occurs at the selected sample site, such as the dorsal aspect of the forearm. The interface of the sample probe of the sample module to the sample site is preferably facilitated with a guide, alignment tool, post, or optical marker.

Tissue Sample

The GTS measures glucose concentration at a tissue sample. Preferably, the GTS accommodates either arm for data collection. When a guide is used, the guide assembly fits both arms. Once the sensor head is inserted into the guide, no additional interaction or adjustment of the guide is required until after the process is complete and the sensor head is removed from guide. Preferably, an aperture of a guide is of equal size with only the curvature being different to accommodate from the 5% female population to the 95% male population.

Construction

The GTS is preferably rugged and semi-portable and the GTS must be approvable by government bodies. Therefore, housing materials and carrying cases are examined, infra.

The preferable housing materials are thermoplastics. Further, the materials used in construction are to be biocompatible and meet the applicable government regulatory body approval. Housing materials preferably withstand a drop from a height of three feet to a hard, non-carpeted floor surface and remain intact. Housing materials, as well as all components used in the GTS, except consumable or expendable items, preferably have a minimum expected useful life of at least five years.

A container is preferably used to facilitate transport and storage of the device and all of its peripherals within short distances. Preferably, this container is made of materials that allow for cleaning with standard medical disinfectants, such as 10% bleach or an alcohol solution. A container is preferably used to ship the instrument and its peripherals through the mail or standard carrier service.

Lamp Replacement

The useful life for the illumination source lamp is preferably at least one year of normal operation. An internal counter, implemented in hardware or software, is optionally used to total lamp hours and/or thermal cycles to determine when the lamp is approaching the end of its useful life. When these counters reach the appropriate values, a message is preferably displayed on the output device that prompts the operator or user to replace the lamp.

Environmental Conditions

This section specifies the range of environmental conditions in which the GTS is expected to function normally. The ambient operating temperature for the device is preferably about 23±5 degrees Celsius. The storage and shipping temperature are preferably from −20° C. to 50° C. at 0% to 100% relative humidity, non-condensing.

Resistance to Liquid-Induced Damage

The GTS is intended to be used in a dry environment, and should not be exposed to liquids. However, the instrument design and materials are such that the unit is, optionally, cleaned, such as with damp soft cotton cloth. Alternatively, cleaning is accomplished with a standard medical disinfectant solution. The GTS is resistant to water-based splashes or spills that are wiped up immediately. Such exposure has no effect on operation and presents no additional shock hazard to the user. The GTS and the sensor head is not intended to be waterproof and should not be immersed in water or other fluids.

Device Labeling

The GTS includes product labeling that assists the user in normal operation. Examples of labeling include:
name and place of business of the manufacturer;
name of the device;
storage and handling;
warnings and precautions;
intended use or purpose;
installation requirements;
theory and functions;
performance and limitations of use;
directions for use;
maintenance;
trouble-shooting; and
technical specifications.

In a second example of the invention, the glucose tracking system is a handheld unit, FIG. 3. The essential elements of the second example are a base module 30 with a spectrograph, a communication bundle 31 having a single fiber optic, a sample module that includes a broadband source 32, and an associated processor for executing an algorithm. Additional components in the handheld unit include one or more of the analyzer components as described in the first example of the invention, supra, e.g. a use port 33, touch-screen LCD 34, backlit display 35, and power switch 36.

In a third example of the invention, the glucose tracking system is an analyzer that includes a base module with a spectrograph, a communication bundle, and a sample module. The analyzer operates at multiple wavelengths, such as in the near infrared from about 1100 to 1900 nm or ranges therein. Additional components in the handheld unit include one or more of the analyzer components as described in the above examples of the invention, supra.

In a fourth example of the invention, the glucose tracking system is an analyzer that includes a base module with a spectrograph, a communication bundle, and a sample module. The communication bundle includes a single fiber optic strand, such as a fiber strand of about 100, 200, 300, or 400 μm. Additional components in the handheld unit include one or more of the analyzer components as described in the above examples of the invention, supra.

The values in the text and figures are exemplary only and are not meant to limit the invention. Although the invention has been described herein with reference to certain preferred embodiments, one skilled in the art will readily appreciate that other applications may be substituted for those set forth herein without departing from the spirit and scope of the present invention. Accordingly, the invention should only be limited by the Claims included below.

1. An apparatus for noninvasive glucose concentration estimation using near-infrared spectroscopy, comprising:
a base module comprising a wavelength separation device and at least one detector;
a sample module, irremovably contractable to a tissue sample, said sample module comprising an illumination source; and
a communication bundle attaching said base module to said sample module, for carrying power to said sample module from said base module, and for carrying an optical signal to said base module from said sample module.

2. The apparatus of claim 1, wherein said wavelength separation device comprises a grating.

3. The apparatus of claim 2, wherein said detector comprises a detector array.

4. The apparatus of claim 3, wherein said illumination source comprises a broadband source.

5. The apparatus of claim 1, wherein said communication bundle carries electrical signals between said base module and said sample module.

6. The apparatus of claim 1, wherein at least a portion of said communication bundle facilitates movement of a coupling fluid.

7. The apparatus of claim 6, where said coupling fluid comprises a fluorocarbon polymer.

8. The apparatus of claim 1, further comprising at least one fiber optic strand, wherein said fiber optic is at least partially contained in said sample module and is at least partially contained in said communication bundle.

9. The apparatus of claim 8, wherein said fiber optic strand is less than 400 micrometers in diameter.

10. The apparatus of claim 9, wherein said fiber optic strand diameter is about 200 to 320 micrometers in diameter.

11. The apparatus of claim 10, wherein said fiber optic strand diameter is about 300 to 320 micrometers in diameter.

12. The apparatus of claim 1, wherein said communication bundle comprises a flexible housing.

13. An apparatus for noninvasive glucose concentration estimation of a tissue sample using near-infrared spectroscopy, comprising:
a base module comprising a grating and a detector array;
a sample module comprising a broadband illumination source; and
a communication bundle coupling said base module to said sample module, for carrying optical signals between said sample module and said base module, and for carrying power to said sample module from said base module.

14. The apparatus of claim 13, wherein said sample module further comprises a reflector.

15. The apparatus of claim 13, wherein said sample module further comprises at least one optical window.

16. The apparatus of claim 15, wherein said optical window comprises at least one of:
a bandpass filter;
a longpass filter;
a shortpass filter; and
a filter used to restrict wavelengths that reach said tissue sample.

17. The apparatus of claim 13, wherein said sample module comprises at least two optical windows.

18. The apparatus of claim 13, wherein said sample module further comprises at least one fiber optic strand.

19. The apparatus of claim 13, wherein said near-infrared spectroscopy comprises wavelengths of about 1100 to 1900 nm.

20. The apparatus of claim 13, wherein said near-infrared spectroscopy comprises wavelengths of about 1200 to 1850 nm.

21. The apparatus of claim 13, wherein said communication bundle facilitates transport of a coupling fluid.

22. The apparatus of claim 21, wherein said coupling fluid comprises an index matching coupling fluid.

23. The apparatus of claim 21, wherein said coupling fluid comprises a perfluorocarbon.

24. The apparatus of claim 13, wherein said base module further comprises a central processing unit used for at least three of: system performance checks, signal filtering, error checking, glucose concentration estimation, and identification of incorrect spectra.

25. An apparatus for noninvasive measurement of glucose concentration through near-infrared spectroscopy, comprising:
a base module comprising a grating and a detector array;
a sample module, securely and removeably attachable to a tissue sample, said sample module comprising an illumination source;
a communication bundle coupling said base module and said sample module; and
a coupling fluid delivery system comprising a reservoir and a fluid path between said reservoir and a location at or proximate to said tissue sample.

26. The apparatus of claim 25, wherein said coupling fluid comprises a fluoropolymer.

27. The apparatus of claim 25, wherein said reservoir is attached or integrated into at least one of said base module and said sample module.

28. The apparatus of claim 25, wherein said reservoir comprises a replaceable cartridge.

29. The apparatus of claim 25, wherein said sample module further comprises a temperature controlled region.

30. The apparatus of claim 29, wherein said temperature controlled region is either of actively or passively controlled.

31. The apparatus of claim 29, wherein said region at least partially encompasses said fluid path.

32. The apparatus of claim 31, wherein said region conductively controls said coupling fluid within a range of about 85 to 100 degrees Fahrenheit as delivered to said sample site.

33. The apparatus of claim 32, where said range comprises about 90 to 95 degrees Fahrenheit.

34. The apparatus of claim 33, wherein said illumination source comprises a broadband source.

35. The apparatus of claim 32, where said base module is housed in a first unit and said sample module is housed in a second unit.

36. An apparatus for near-infrared noninvasive glucose concentration determination, comprising:
a base module in a first housing, wherein said base module comprises a wavelength selection device and at least one detection element;
a sample module in a second housing, said sample module comprising an illumination source; and
a communication bundle for carrying optical and/or electrical signals between said sample module and said base module, and for carrying power to said sample module from said base module.

37. The apparatus of claim 36, wherein said wavelength selection device comprises a grating.

38. The apparatus of claim 37, wherein said detection element comprises an array of detectors.

39. The apparatus of claim 38, wherein said array of detectors comprises InGaAs detectors.

40. The apparatus of claim 36, wherein said base module further comprises an output element.

41. The apparatus of claim 40, wherein said output element comprises a graphical output unit.

42. The apparatus of claim 41, wherein said graphical output unit comprises a liquid crystal display.

43. The apparatus of claim 42, wherein said liquid crystal display further comprises a touch screen interface.

44. The apparatus of claim 36, wherein said illumination source comprises a gas-filled lamp.

45. The apparatus of claim 36, wherein said illumination source comprises a tungsten filament lamp.

46. The apparatus of claim 36, wherein said glucose concentration determination is in the range of about 50 to 350 mg/dL.

47. The apparatus of claim 36, wherein out of range glucose concentrations are reported with an error code.

48. The apparatus of claim 36, wherein said sample module weighs less than about 60 grams.

49. The apparatus of claim 48, wherein said sample module weighs about 27 to 50 grams.

50. The apparatus of claim 49, wherein said sample module weighs about 30 to 40 grams.

51. An apparatus for noninvasive glucose level determination from a tissue sample, comprising:
a base module comprising a grating and a detector array;
a sample module, said sample module comprising an illumination source, wherein said sample module does not contain an operative near-infrared detector; and
a communication bundle for carrying optical and/or electrical signals between said sample module and said base module, and for carrying power to said sample module from said base module.

52. The apparatus of claim 51, further comprising a guide replaceably attached to said sample module and said tissue sample.

53. The apparatus of claim 52, further comprising an occlusion plug, wherein said plug fits inside said guide.

54. The apparatus of claim 53, wherein said plug further comprises means for photostimulation of said tissue sample.

55. The apparatus of claim 53, further comprising one or more light emitting diodes, wherein light from said diode photostimulates at least one of said tissue sample and a region about said tissue sample.

56. The apparatus of claim 55, where said light emitting diode emits light about 890 or 910 nm.

57. An analyzer for noninvasively estimating tissue and/or blood glucose concentration, comprising:
a base module comprising a grating and a detector array;
a sample module, said sample module comprising an illumination source, wherein said base module and said sample module are integrated together into a single unit; and
a coupling fluid delivery system comprising a reservoir and at least one delivery path between said reservoir and a sample site.

58. The apparatus of claim 57, wherein said analyzer operates on the left or right arm of a subject.

59. The apparatus of claim 58, wherein said analyzer is battery powered.

60. The apparatus of claim 59, wherein said analyzer applies less than about 60 grams of weight to a sample.

61. The apparatus of claim 60, wherein said analyzer applied between about 27 and 50 grams of weight to a sample.

62. A method for using a noninvasive glucose concentration analyzer on a subject, comprising the steps of:
powering on the analyzer, wherein said analyzer comprises a sample module in a first housing and a base module in a second housing and where a communication bundle interfaces said sample module and said base module;
preparing a sample, wherein preparing said sample comprises cleaning a sample site and applying a guide;
collecting a sample spectrum of said sample site; and
determining a glucose concentration from said sample spectrum.

63. The method of claim 62, further comprising the steps of:
performing an analyzer self test.

64. The method of claim 63, wherein preparing said sample further comprises the steps of:
shaving said sample site.

65. The method of claim 62, further comprising performing the step of:
photostimulation on said sample site, to minimize differences in glucose concentration between said sample site and a vascular region of said subject.

66. The method of claim 62, wherein said collecting and determining steps are repeated to generate a glucose concentration profile as a function of time.

67. The method of claim 65, wherein said collecting, determining and photostimulation steps are repeated to generate a glucose concentration profile as a function of time.

68. A method for determining a noninvasive glucose concentration from a subject sample site, comprising the steps of:
turning on an analyzer;
collecting a reference glucose concentration;
preparing a sample site by applying a guide about said sample site; and
by performing photostimulation on said sample site;
collecting a noninvasive sample spectrum of said sample site;
determining a first glucose concentration estimation from said sample spectrum;
recording a final glucose concentration using said first glucose concentration and said reference glucose concentration; and
correcting bias by using said reference glucose concentration.

69. The method of claim 68, wherein said collecting, determining and recording steps are repeated to yield a set of final glucose concentrations as a function of time.
